(19) United States
(12) Patent Application Publication
(10) Pub. No.: US 2009/0169477 A1
(43) Pub. Date: Jul. 2, 2009

(54) DELAYING OR PREVENTING ONSET OF MULTIPLE SCLEROSIS

(76) Inventors: Michael Panzara, Winchester, MA (US); Marco Rizzo, Guilford, CT (US)

Correspondence Address: LOWRIE, LANDO & ANASTASI, LLP;B2047, ONE MAIN STREET, SUITE 1100, CAMBRIDGE, MA, 02142, US

(21) Appl. No.: 11/720,716

(22) PCT Filed: Dec. 2, 2005
(86) PCT No.: PCT/US05/43980

§ 371 Date: Feb. 25, 2009

Related U.S. Application Data
(60) Provisional application No. 60/633,022, filed on Dec. 3, 2004.

(51) Int. Cl. A61K 49/00; A61K 39/395

(52) U.S. Cl. 424/9.1; 424/130.1; 424/133.1

(57) ABSTRACT

Methods of treating persons at risk for relapsing MS are described.

### RELATED APPLICATIONS

This application claims the benefit of U.S. Provisional Application No. 60/633,022 filed Dec. 3, 2005, the entire contents of which are hereby incorporated by reference herein.

### BACKGROUND

Multiple sclerosis (MS) is a chronic, multifocal, demyelinating, autoimmune disease of the central nervous system. Over 2 million people have MS worldwide with 400,000 in the US. Approximately 80% of MS patients have a relapsing form with >80% of these patients progressing to secondary progressive MS in 25 years.

### SUMMARY OF THE INVENTION

In one aspect, the invention features a method of treating a subject at risk for multiple sclerosis (MS), e.g., at risk of progressive MS or relapsing MS. The method includes administering to the subject a VLA-4 blocking agent, e.g., a VLA-4 binding antibody (e.g., a full length VLA-4 binding antibody or VLA-4 binding antibody fragment). In one embodiment, the method can prevent or delay (e.g., for at least one year, 2 years, 3 years, 4 years, 5 years, 10 years or more) the onset of clinical manifestations of MS (e.g., relapsing remitting MS) or can minimize the severity of a subsequent (e.g., a second) clinical manifestation. In one embodiment, the subject has had fewer than two clinical episodes of focal neurologic deficit.

In one embodiment, the subject has experienced one clinical episode of focal neurologic deficit. The neurologic deficit can be evidenced by, e.g., one or more symptoms, such as weakness of one or more extremities, paralysis of one or more extremities, tremor of one or more extremities, uncontrollable muscle spasticity, sensory loss or abnormality, decreased coordination, loss of balance, loss of ability to think abstractly, loss of ability to generalize, difficulty speaking, and difficulty understanding speech.

The VLA-4 blocking agent can be administered within 6, 4, 3, 2, or 1 weeks of the clinical episode.

In another embodiment, the subject is indicated as being at risk for multiple sclerosis by detection of neurological damage. For example, the subject can be evaluated, e.g., using a cranial scan, e.g., by a radiographic scan, a computed tomography (CT) scan, or a magnetic resonance imaging (MRI) scan. Detection of physical evidence of brain tissue inflammation or myelin sheath damage can indicate the subject for treatment in the absence of a clinical episode or in conjunction with one clinical episode. In another example, the subject can be treated if at least two, three, five, ten, fifteen, twenty, or twenty-five individual brain lesions or scars (e.g., those greater than or equal to 1.5 or 3 mm in size) are detectable, e.g., by MRI.

In another embodiment, the subject is indicated as being at risk for multiple sclerosis by a biochemical or physiological criterion, e.g., in the absence of a clinical episode or in conjunction with one clinical episode. For example, presence of serum antibodies against one or both of myelin oligodendrocyte glycoprotein (MOG) and myelin basic protein (MBP) can indicate that the subject is at risk.

Subjects can also be indicated for treatment by a combination of criteria described herein. A subject can be given a VLA-4 blocking agent, e.g., if the subject has at least one, two, three, four, or five risk factors for MS, e.g., risk factors described herein. For example, a subject who has experienced one clinical episode of neurologic deficit and who has a detectable neurological damage or indicative biochemical or physiological criteria can be treated. In another example, the subject has not experienced a clinical episode of neurologic deficit, but is indicated by detectable neurological damage or indicative biochemical or physiological criteria. For example, a subject who has not experienced a clinical episode of focal neurologic deficit, may be indicated for treatment by one or more of the following characteristics: (a) has a plurality of brain lesions or scars greater than or equal to 3 mm in size detectable by cranial scan, (b) has serum antibodies against one or both of myelin oligodendrocyte glycoprotein (MOG) and myelin basic protein (MBP), (c) has increased levels of CSF IgG compared to a control, and (d) has elevated levels of myelin basic protein (MBP) compared to a control.

In another embodiment, the subject has a family history of multiple sclerosis, e.g., at least one parent, sibling, or grandparent who has multiple sclerosis. In one embodiment, the subject has had one acute isolated demyelinating event, e.g., an event involving the optic nerve, spinal cord or cerebellum. In another embodiment, the subject has a clinically silent feature of multiple sclerosis. For example, the subject has at least one, two, five, or ten clinically silent brain MRI lesions greater than or equal to 3 mm in size. In one embodiment, the subject has transverse myelitis or optic neuritis.

The subject can in some cases be evaluated for exclusion of pathologies associated with disorders other than MS. For example, the subject can be determined not to have metabolic, vascular, collagen-vascular, infectious, and/or neoplastic disease that may cause neurologic deficit. For example, the subject is determined not to have a stroke, CNS lymphoma, brainstem glioma, or a lysosomal storage disease.

In one embodiment, at least at point of initial administration, the subject has an EDSS score of less than 3, 2, 1.5, or 1.

In one embodiment, the subject is an adult, e.g., a subject whose age is greater or equal to 16, 18, 19, 20, 24, or 30 years. For example, the subject is between 19 and 40 years of age. The subject can be female or male. The subject can be administered doses of the VLA-4 blocking agent for greater than 14 weeks, e.g., greater than six or nine months, greater than 1, 1.5, or 2 years, e.g., at generally regular intervals.

In one implementation, the method includes before the administering step, selecting a subject as being at risk for MS on the basis of one or more of: (a) cranial scan having evidence of myelin sheath damage, (b) presence of serum antibodies against one or both of MOG and MBP, (c) presence of increased levels of CSF IgG, (d) presence of elevated levels of MBP, and (e) occurrence of one clinical episode of focal neurologic deficit.

In one embodiment, the VLA-4 blocking agent includes a VLA-4 binding antibody, e.g., a full length antibody such as an IgG1, IgG2, IgG3, or IgG4. The antibody can be effectively human, human, or humanized. The VLA-4 binding antibody can inhibit VLA-4 interaction with a cognate ligand of VLA-4, e.g., VCAM-1. The VLA-4 binding antibody binds to at least the a chain of VLA-4, e.g., to the extracellular domain of the α4 subunit. For example, the VLA-4 binding antibody recognizes epitope B (e.g., B1 or B2) on the α chain of VLA-4. The VLA-4 binding antibody may compete with natalizumab, HP1/2, or another VLA-4 binding antibody described herein for binding to VLA-4. In a preferred embodiment, the VLA-4 binding antibody is natalizumab or includes the heavy chain and light chain variable domains of natalizumab.

Early treatment can, for example, prevent the development of disability over the long term, decrease T2 and Gd+ lesions over time, prevent the development of secondary progressive MS, and/or prevent the development of permanent brain tissue injury (e.g., as detected on MRI).

In another aspect, the disclosure features a method that includes: evaluating a subject or receiving information about an evaluation of a subject; and administering to the subject a VLA-4 binding antibody if the evaluation indicates that the subject is at risk for MS. In one embodiment, the method includes: performing a scan on a subject, and administering to the subject a VLA-4 blocking agent if the scan shows evidence of a clinically silent feature of MS (e.g., early MS). Examples of clinically silent features include brain tissue inflammation or myelin sheath damage, e.g., the presence of Gd+, T1 or T2 lesions in the absence of a clinical episode of neurologic deficit. Other exemplary evaluations include evaluations for risk factors described herein. The subject can be evaluated for at least one, two, three, or four risk factors. The subject can be administered the VLA-4 blocking agent if at least one, two, three, or four risk factors are detected.

In another aspect, the disclosure features a method that includes: identifying a subject having a monophasic demyelinating disorder; and administering to the subject a VLA-4 binding antibody, e.g., in an amount effective to treat the disorder. For example, the subject has a disorder that is not clinically definite multiple sclerosis. The subject can have, e.g., transverse myelitis, optic neuritis, or acute disseminated encephalomyelitis (ADEM).

Definitions

A “neurologic deficit” is a decrease in a function of the central nervous system. Examples include inability to speak, decreased sensation, loss of balance, weakness, cognitive dysfunction, visual changes, abnormal reflexes, and problems walking. A “focal neurologic deficit” affects either a specific location (such as the left face, right face, left arm, right arm) or a specific function (for example, speech may be affected, but not the ability to write). When referring to a neurologic deficit, the term “clinical episode” means a neurologic deficit that lasts for hours, days or weeks (but from which partial or complete recovery can take place) and that is directly observable by outward physical signs of a patient, as distinguished from being observable only through a laboratory test or imaging of internal body tissues. A clinical neurologic deficit is typically determined by a medical history and/or a physical neurological exam.

The term “treating” refers to administering a therapy in an amount, manner, and/or mode effective to improve a condition, symptom, or parameter associated with a disorder or to prevent or reduce progression of a disorder, either to a statistically significant degree or to a degree detectable to one skilled in the art. An effective amount, manner, or mode can vary depending on the subject and may be tailored to the subject.

A “cranial scan” is a technique for examining and obtaining an image of the brain in a living person. Examples include CT scans and MRI scans.

The term “biologic” refers to a protein-based therapeutic agent.

A “VLA-4 binding agent” refers to any compound that binds to VLA-4 integrin with a Kd of less than 10⁻⁶ M. An example of a VLA-4 binding agent is a VLA-4 binding protein, e.g., an antibody such as natalizumab.

A “VLA-4 antagonist” refers to any compound that at least partially inhibits an activity of a VLA-4 integrin, particularly a binding activity of a VLA-4 integrin or a signaling activity, e.g., ability to transduce a VLA-4 mediated signal. For example, a VLA-4 antagonist may inhibit binding of VLA-4 to a cognate ligand of VLA-4, e.g., a cell surface protein such as VCAM-1, or to an extracellular matrix component, such as fibronectin or osteopontin. A typical VLA-4 antagonist can bind to VLA-4 or to a VLA-4 ligand, e.g., VCAM-1 or an extracellular matrix component, such as fibronectin or osteopontin. A VLA-4 antagonist that binds to VLA-4 may bind to either the α4 subunit or the β1 subunit, or to both. A VLA-4 antagonist may also interact with other α4 subunit containing integrins (e.g., α,4β7) or with other β1 containing integrins. A VLA-4 antagonist may bind to VLA-4 or to a VLA-4 ligand with a K_{d} of less than 10⁻⁶, 10⁻⁷, 10⁻⁸, 10⁻⁹, or 10⁻¹⁰ M.

As used herein, the term “antibody” refers to a protein that includes at least one immunoglobulin variable region, e.g., an amino acid sequence that provides an immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab fragments, F(ab′)₂, a Fd fragment, a Fv fragments, and dAb fragments) as well as complete antibodies, e.g., intact immunoglobulins of types IgA, IgG, IgE, IgD, IgM (as well as subtypes thereof). The light chains of the immunoglobulin may be of types kappa or lambda. In one embodiment, the antibody is glycosylated. An antibody can be functional for antibody-dependent cytotoxicity and/or complement-mediated cytotoxicity, or may be non-functional for one or both of these activities.

The VH and VL regions can be further subdivided into regions of hypervariability, termed “complementarity determining regions” (“CDR”), interspersed with regions that are more conserved, termed “framework regions” (“FR”). The extent of the framework region and CDR's has been precisely defined (see, Kabat, E. A., et al. (1991) Sequences of Proteins of Immunological Interest, Fifth Edition, US Department of Health and Human Services, NIH Publication No. 91-3242, and Chothia et al. (1987) J. Mol. Biol. 196:901-917). Kabat definitions are used herein. Each VH and VL is typically composed of three CDR's and four FR's, arranged from amino-terminus to carboxyl-terminus in the following order: FR1, CDR1, FR2, CDR2, FR3, CDR3, FR4.

An “immunoglobulin domain” refers to a domain from the variable or constant domain of immunoglobulin molecules. Immunoglobulin domains typically contain two β-sheets formed of about seven β-strands, and a conserved disulphide bond (see, e.g., Williams et al., 1988 Ann. Rev Immunol. 6:381-405).

As used herein, an “immunoglobulin variable domain sequence” refers to an amino acid sequence that can form the structure of an immunoglobulin variable domain. For example, the sequence may include all or part of the amino acid sequence of a naturally-occurring variable domain. For example, the sequence may omit one, two or more N- or C-terminal amino acids, internal amino acids, may include one or more insertions or additional terminal amino acids, or may include other alterations. In one embodiment, a polypeptide that includes immunoglobulin variable domain sequence can associate with another immunoglobulin variable domain sequence to form a target binding structure (or “antigen binding site”), e.g., a structure that interacts with VLA-4.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains. The heavy and light immunoglobulin chains can be connected by disulfide bonds. The heavy chain constant region typically includes three constant domains, CH1, CH2 and CH3. The light chain constant region typically includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

One or more regions of an antibody can be human or effectively human. For example, one or more of the variable regions can be human or effectively human. For example, one or more of the CDRs can be human, e.g., HC CDR1, HC CDR2, HC CDR3, LC CDR1, LC CDR2, and LC CDR3. Each of the light chain CDRs can be human. HC CDR3 can be human. One or more of the framework regions can be human, e.g., FR1, FR2, FR3, and FR4 of the HC or LC. In one embodiment, all the framework regions are human, e.g., derived from a human somatic cell, e.g., a hematopoietic cell that produces immunoglobulins or a non-hematopoietic cell. One or more of the constant regions can be human or effectively human. In another embodiment, at least 70, 75, 80, 85, 90, 92, 95, or 98% of the framework regions (e.g., FR1, FR2, and FR3, collectively, or FR1, FR2, FR3, and FR4, collectively) or the entire antibody can be human or effectively human. For example, FR1, FR2, and FR3collectively can be at least 70, 75, 80, 85, 90, 92, 95, 98, or 99% identical to a human sequence encoded by a human germline segment.

An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

A “humanized” immunoglobulin variable region is an immunoglobulin variable region that is modified to include a sufficient number of human framework amino acid positions such that the immunoglobulin variable region does not elicit an immunogenic response in a normal human. Descriptions of “humanized” immunoglobulins include, for example, U.S. Pat. No. 6,407,213 and U.S. Pat. No. 5,693,762. In some cases, humanized immunoglobulins can include a non-human amino acid at one or more framework amino acid positions.

All or part of an antibody can be encoded by an immunoglobulin gene or a segment thereof. Exemplary human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 Kd or 214 amino acids) are encoded by a variable region gene at the NH2-terminus (about 110 amino acids) and a kappa or lambda constant region gene at the COOH-terminus. Full-length immunoglobulin “heavy chains” (about 50 Kd or 446 amino acids), are similarly encoded by a variable region gene (about 116 amino acids) and one of the other aforementioned constant region genes, e.g., gamma (encoding about 330 amino acids).

The term “antigen-binding fragment” of a full length antibody refers to one or more fragments of a full-length antibody that retain the ability to specifically bind to a target of interest, e.g., VLA-4. Examples of binding fragments encompassed within the term “antigen-binding fragment” of a full length antibody include (i) a Fab fragment, a monovalent fragment consisting of the VL, VH, CL and CH1 domains; (ii) a F(ab′)₂ fragment, a bivalent fragment including two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al, (1989) Nature 341:544-546), which consists of a VH domain; and (vi) an isolated complementarity determining region (CDR) that retains functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See, e.g., Bird et al. (1988) Science 242:423-426; and Huston et al. (1988) Proc. Natl. Acad. Sci. USA 85:5879-5883.

### DETAILED DESCRIPTION

### Multiple Sclerosis

A diagnosis of MS can be made on the basis of multiple clinical episodes of focal neurologic deficit or, alternatively, on the basis of a clinical episode of focal neurologic deficit separated in space and time from supporting evidence of neurologic damage from ancillary tests such as MRI (McDonald et al., Ann. Neurol., 2001, 50:121-7). The McDonald criteria allow for the second attack in time to be defined by a new lesion appearing on MRI. Also, the MacDonald criteria allow the dissemination in space to be established on the basis of either 9 typical white matter lesions or 1 enhancing lesion on MRI. The initial clinical presentation may vary, and it may include somatic sensory changes, optic neuritis, or weakness. For a true clinical diagnosis, at least two neurologic impairments must be observed, and these must separated by both anatomy and time. Further, impairment must be compatible with impairment found in patients with MS, which typically means that the duration of deficit is days to weeks. The methods described herein can be used, e.g., to prevent or reduce progression to clinically definite MS or relapsing MS.

The overall risk of developing MS (e.g., relapsing MS) after a single episode of neurologic impairment is estimated to be as low as 12% (Beck et al., 1993, N. Engl. J. Med. 329:1764-1769) to as high as 58% (Rizzo et al., 1988, Neurology 38:185-90). MRI has been proven to be the most useful investigation for predicting the progression to MS. In a 10-year follow-up study of patients with a clinically isolated event, 45 of 54 patients (83%) with abnormal MRI findings went on to develop clinical MS, whereas only 3 of 27 patients with normal MRI findings developed MS (O'Riordan et al., 1998, Brain 121(Pt 3):495-503).

Tintoré et al. followed up 70 patients for an average of 28.3 months after an isolated neurologic event and compared various MRI criteria for the diagnosis MS, as defined by Paty et al., Fazekas et al., and Barkhof et al. (Tintoré et al., 2000, AJNR Am. J. Neuroradiol. 21:702-706; Paty et al., 1988, Neurology 38:180-185; Fazekas et al., 1988, Neurology 38:1822-1825; Barkhof et al., 1997, Brain 120:2059-2069). With the method of Paty et al., which requires 3 or 4 lesions (1 of which is periventricular), the authors reported a sensitivity of 86% but a specificity of only 54%.

The criteria of Fazekas et al. resulted in the same sensitivity and specificity. These criteria require 3 lesions with 2 of the 3 following characteristics: infratentorial location, periventricular location, and lesion greater than 6 mm. The criteria of Barkhof require 1 infratentorial lesion, 1 juxtacortical lesion, 3 periventricular lesions, and either 1 gadolinium-enhanced lesion or more than 9 lesions on T2-weighted MRIs. These criteria resulted in a sensitivity of 73% and a specificity of 73%. Thus, as the MRI criteria become more stringent in the diagnosis of MS, specificity increases at the expense of decreasing sensitivity.

### Clinically Isolated Syndrome (CIS) and Monophasic Inflammatory Disorders

Single incidents of neurological impairment are indicative of a patient whose condition can be improved with a VLA-4 blocking agent. Clinically isolated syndrome (CIS) refers to the detection of a single clinical episode of demyelination or other monophasic CNS inflammatory disorder (e.g., Spinal Cord Syndrome, Brainstem/Cerebellar Syndrome, and others described below).

Frohman et al. (2003) Neurology. 2003 Sep 9;61(5):602-11 report that, in subjects with CIS, three or more white matter lesions on a T2-weighted MRI scan (especially if one of these lesions is located in the periventricular region) is a very sensitive predictor (>80%) of the subsequent development of CDMS within the next 7 to 10 years. The presence of two or more gadolinium (Gd)-enhancing lesions at baseline and the appearance of either new T2 lesions or new Gd enhancement on follow-up scans are also highly predictive of the subsequent development of CDMS in the near term. Dalton et al. (2004) Brain 127(Pt 5):1101-7, report that the mean decrease in grey matter fractional volume (GMF, as a fraction of total intracranial volume) is an indicator of CIS subjects that are likely to progress to MS.

A VLA-4 blocking agent described herein can be administered to a subject who has CIS, e.g., in an amount effective to delay onset of a subsequent episode, e.g., by at least one year, two years, three years or more. The agent can be administered to a CIS subject who also has at least one, two, or three white matter lesions on a T2-weighted MRI scan, one or more of which can be located in the periventricular region. The method can further include periodically evaluating the subject, e.g., by MRI scanning, to determine the number of MRI-detectable lesions or a change in grey matter fractional volume.

A VLA-4 blocking agent described herein can also be administered in a therapeutically effective amount to a subject who has a monophasic CNS inflammatory disorder, e.g., transverse myelitis, optic neuritis, or acute disseminated encephalomyelitis (ADEM).

Spinal Cord Syndrome

Subjects with spinal cord syndrome have a spinal MRI that is consistent with a demyelinating event and have a symptom of myelopathy, e.g., one or more of the following: (a) Brown-Sequard syndrome; (b) crural and/or brachial paresis or plegia (unilateral or bilateral); (c) urinary incontinence or retention; (d) fecal incontinence or retention; (e) paroxysmal dystonia; (f) Lhermitte's phenomena.

Brainstem/Cerebellar Syndrome

Subjects with brainstem/cerebellar syndrome have a neurological examination abnormality consistent with the subject's symptoms as determined by a skilled neurologist. Symptoms include at least 2 of the following: (a) vertigo, (b) trigeminal neuralgia, (c) internuclear ophthalmoparesis (plegia), (d) nystagmus, (e) oscillopsia and diplopia, (f) conjugate or dysconjugate gaze palsies (paresis), (g) crossed motor syndrome, (h) crossed sensory syndrome, (i) hemifacial spasm, (j) ataxia, (k) tremor, (l) dysarthria.

Transverse Myelitis/Partial Myelitis

Transverse myelitis is a neurological disorder caused by inflammation across both sides of one level, or segment, of the spinal cord. Attacks of inflammation can damage or destroy myelin, interrupting communications between the nerves in the spinal cord and the rest of the body. Symptoms of transverse myelitis include a loss of spinal cord function over several hours to several weeks. What can begin as a sudden onset of lower back pain, muscle weakness, or abnormal sensations in the toes and feet can rapidly progress to more severe symptoms, including paralysis, urinary retention, and loss of bowel control. Although some patients can recover from transverse myelitis with minor or no residual problems, others can suffer permanent impairments that affect their ability to perform ordinary tasks of daily living. Most patients have only one episode of transverse myelitis. A small percentage may have a recurrence.

An acute, rapidly progressing form of transverse myelitis sometimes signals the first attack of multiple sclerosis (MS); however, studies indicate that most people who develop transverse myelitis do not go on to develop MS. Patients with transverse myelitis can nonetheless be screened for MS because patients with this diagnosis can require different treatments. Partial myelitis can more commonly be predictive of MS.

Optic Neuritis

Optic Neuritis is an inflammation, with accompanying demyelination, of the optic nerve (Cranial Nerve II) serving the retina of the eye. It can present with any one or more of the following symptoms: blurring of vision, loss of visual acuity, loss of some or all color vision, complete or partial blindness and pain behind the eye. Presentation is unilateral (in one eye) in 70% of cases. Optic neuritis is an initial manifestation (first attack) of MS in about 20% of MS patients. Diagnostic tests for optic neuritis include visually evoked potential (VEP) and visually evoked response (VER) tests, which detect the speed of nerve transmission along the optic nerve.

A patient having optic neuritis can be identified by the presence of one or more (preferably all) of the following: (a) unilateral (as opposed to bilateral) optic neuritis; (b) history of sudden vision loss usually accompanied by pain; (c) evidence of optic nerve dysfunction (e.g., presence of a relative afferent pupillary effect (RAPD) and a visual filed defect in the involved eye); (d) a normal or swollen (but not pale) optic disc in the affected eye; (e) no more than trace macular exudates, iritism or vitreous cells; (f) absence of any other finding on examination to explain the visual symptoms.

Acute Disseminated Encephalomyelitis (ADEM)

ADEM is a monophasic demyelinating disorder of the CNS that is generally preceded by a viral syndrome or vaccinations. It can be associated with loss of myelin, with relative sparing of the axon. Perivenular lymphocytic and mononuclear cell infiltration and demyelination can often be seen.

### Risk of MS

The etiology of multiple sclerosis is complex. One or more factors may contribute to risk for multiple sclerosis, such factors include those presently known and ones yet to be determined to a statistically significant impact by those skilled in the art.

The manifestation of a clinically isolated syndrome or monophasic inflammatory disorder is one event that can indicate that a subject is at risk for multiple sclerosis. Other examples of risk factors can include geographic location, environmental factors, and gene polymorphism. Environmental factors can include prior exposure to pharmaceuticals and vaccines. For example, Hernan et al. (2004, Neurology 63:838-42) reported that a vaccination for hepatitis B could contribute to risk for multiple sclerosis.

Genetic factors also can contribute to risk for multiple sclerosis. Familial aggregation is well documented. Risk for multiple sclerosis is also increased about 2-40 fold compared to the general population if a genetic family member has multiple sclerosis. For example, a 20-fold increase in risk can apply to monozygotic twins.

MS1, the major histocompatibility complex. The HLA-DR2 haplotype (DRB1*1501 DQB1*0602) within the major histocompatibility complex (MHC) on the short arm of chromosome 6 is the strongest genetic effect identified in MS, and has consistently demonstrated both linkage and association in family and case-control studies. Olerup et al (1991) Tissue Antigens 38:1-15. In addition, MS also has been associated with certain Human Leukocyte Antigen (HLA) haplotypes, particularly the DR2, DR(1*1501), DQ(1*602), DQA102 and the DW2 haplotypes. Genomic screens have shown some support for linkage to this region, and a meta-analysis of all four genomic screens identified 19q13 as the second most significant region after the MHC (Barcellos et al., (1997) JAMA 278:1256-1271; and Pericak-Vance et al., (2001) Neurogenetics 3:195-201).

Bilinska et al. report that a particular SNP in the first exon of the CTLA-4 gene is associated with MS (Acta Neurol Scand. 2004 July; 110(1):67-71). Other genetic loci that can modulate the risk for multiple sclerosis include the gene that encodes ApoE. See, e.g., Schmidt et al., Am. J. Hum. Genet. (2002) 70:708-717.

Geographic and environmental factors can also contribute to risk for multiple sclerosis. For example, Schiffer et al. ((2001) Arch Environ Health. 56(5):389-95) reported a cluster of multiple sclerosis (MS) cases in a small, north-central Illinois community that was the site of significant environmental heavy-metal exposure from a zinc smelter. Pugliatti et al. (Neurology. (2002) 58(2):277-82) found uneven distribution of multiple sclerosis in Sardinia.

### Detection of Exemplary Risk Factors

Cerebrospinal fluid examination can be used to detect risk for MS. For example, one factor is indicated by increased CSF IgG levels, e.g., relative to baseline or to matched normal individuals, or by an elevated ratio of CSF IgG to CSF albumin. See, e.g., Perkin et al. (1983) J Neurol Sci. 60(3):325-36. For example, abnormal ratios can be indicated by an IgG index of greater than or equal to 0.7. The presence of discrete IgG oligoclonal bands by immunofixation electrophoresis can also be indicative for risk for MS.

Antibodies to MOG and MBP can be detected by contacting the serum of a subject with recombinant versions of these proteins. Human recombinant MOG Ig-domain and human myelin derived MBP can be prepared, e.g., according to Reindl et al. (1999) Brain 122: 2047-2056. For example, 1 mg recombinant MOG-Ig or 2 mg MBP can be electrophoresed on an SDS-PAGE gel, and transferred to nitrocellulose or nytran membranes. The membranes can then be blocked with 2% milk powder in phosphate buffered saline (PBS) with 0.05% Tween-20 (PBS-T). The membranes are then contacted with diluted sera (1:1000 for IgG; 1:200 for IgM or IgA, in 2% milk powder in PBS-T) from a subject. The membranes are then washed and evaluated using a secondary antibody, e.g., alkaline phosphatase conjugated anti-human IgG, IgM or IgA (for example, all 1:5000; G6907, G5204 or G5415; all Axell, Westbury, USA) for 1 h at room temperature. After washing, the secondary antibody can be detected using an appropriate alkaline phosphatase detection system (e.g., p-nitro blue tetrazolium chloride and 5-bromo-4-chloro-3-indolyl phosphate (both Roche Molecular Diagnostics, Mannheim, Germany)). If the secondary antibody is coupled to other detection agents, then the protocol can be modified accordingly. See, e.g., Soderstrom et al., Neurology (1998) 50:708-14.

Visual evoked potential examinations can also be used to identify a risk factor for MS. See, e.g., Cuypers et al., (1995) Doc Ophthalmol. 90(3):247-57.

### VLA-4 Binding Antibodies

Natalizumab, an α4 integrin binding antibody, inhibits the migration of leukocytes from the blood to the central nervous system. Natalizumab binds to VLA-4on the surface of activated T-cells and other mononuclear leukocytes. It can disrupt adhesion between the T-cell and endothelial cells, and thus prevent migration of mononuclear leukocytes across the endothelium and into the parenchyma. As a result, the levels of proinflammatory cytokines can also be reduced.

Natalizumab can decrease the number of brain lesions and clinical relapses in patients with relapsing remitting multiple sclerosis and relapsing secondary-progressive multiple sclerosis. Natalizumab can be safely administered to patients with multiple sclerosis when combined with interferon β-1a (IFNβ-1a) therapy. Other VLA-4binding antibodies can have these or similar properties

Natalizumab and related VLA-4 binding antibodies are described, e.g., in U.S. Pat. No. 5,840,299. Monoclonal antibodies 21.6 and HP1/2 are exemplary murine monoclonal antibodies that bind VLA-4. Natalizumab is a humanized version of murine mAb 21.6 (see, e.g., U.S. Pat. No. 5,840,299). A humanized version of HP1/2has also been described (see, e.g., U.S. Pat. No. 6,602,503). Several additional VLA-4binding monoclonal antibodies, such as HP2/1, HP2/4, L25 and P4C2, are described (e.g., in U.S. Pat. No. 6,602,503; Sanchez-Madrid et al., 1986 Eur. J. Immunol., 16:1343-1349; Hemler et al., 1987 J. Biol. Chem. 2:11478-11485; Issekutz and Wykretowicz, 1991, J. Immunol., 147: 109 (TA-2 mab); Pulido et al., 1991 J. Biol. Chem., 266(16):10241-10245; and U.S. Pat. No. 5,888,507).

Some VLA-4 binding antibodies recognize epitopes of the α4 subunit that are involved in binding to a cognate ligand, e.g., VCAM-1 or fibronectin. Many such antibodies inhibit binding to cognate ligands (e.g., VCAM-1 and fibronectin binding).

Many useful VLA-4 binding antibodies interact with VLA-4 on cells, e.g., lymphocytes, but do not cause cell aggregation. However, other anti-VLA-4 binding antibodies have been observed to cause such aggregation. HP1/2 does not cause cell aggregation. The HP1/2 MAb (Sanchez-Madrid et al., 1986) has an extremely high potency, blocks VLA-4 interaction with both VCAM-1 and fibronectin, and has the specificity for epitope B on VLA-4. This antibody and other B epitope-specific antibodies (such as B1 or B2 epitope binding antibodies; Pulido et al., 1991, supra) represent one class of useful VLA-4 binding antibodies.

An exemplary VLA-4 binding antibody has one or more CDRs, e.g., all three HC CDRs and/or all three LC CDRs, of a particular antibody disclosed herein, or CDRs that are, in sum, at least 80, 85, 90, 92, 94, 95, 96, 97, 98, 99% identical to such an antibody, e.g., natalizumab. In one embodiment, the H1 and H2 hypervariable loops have the same canonical structure as those of an antibody described herein. In one embodiment, the L1 and L2 hypervariable loops have the same canonical structure as those of an antibody described herein.

In one embodiment, the amino acid sequence of the HC and/or LC variable domain sequence is at least 70, 80, 85, 90, 92, 95, 97, 98, 99, or 100% identical to the amino acid sequence of the HC and/or LC variable domain of an antibody described herein, e.g., natalizumab. The amino acid sequence of the HC and/or LC variable domain sequence can differ by at least one amino acid, but no more than ten, eight, six, five, four, three, or two amino acids from the corresponding sequence of an antibody described herein, e.g., natalizumab. For example, the differences may be primarily or entirely in the framework regions.

The amino acid sequences of the HC and LC variable domain sequences can be encoded by a sequence that hybridizes under high stringency conditions to a nucleic acid sequence described herein or one that encodes a variable domain or to a nucleic acid encoding an amino acid sequence described herein. In one embodiment, the amino acid sequences of one or more framework regions (e.g., FR1, FR2, FR3, and/or FR4) of the HC and/or LC variable domain are at least 70, 80, 85, 90, 92, 95, 97, 98, 99, or 100% identical to corresponding framework regions of the HC and LC variable domains of an antibody described herein. In one embodiment, one or more heavy or light chain framework regions (e.g., HC FR1, FR2, and FR3) are at least 70, 80, 85, 90, 95, 96, 97, 98, or 100% identical to the sequence of corresponding framework regions from a human germline antibody.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences.

As used herein, the term “hybridizes under high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. High stringency hybridization conditions include hybridization in 6×SSC at about 45° C., followed by one or more washes in 0.2×SSC, 0.1% SDS at 65° C., or substantially similar conditions.

Antibodies can be tested for a functional property, e.g., VLA-4 binding, e.g., as described in U.S. Pat. No. 6,602,503.

### Antibody Generation

Antibodies that bind to VLA-4 can be generated by immunization, e.g., using an animal. All or part of VLA-4 can be used as an immunogen. For example, the extracellular region of the α4 subunit can be used as immunogen. In one embodiment, the immunized animal contains immunoglobulin producing cells with natural, human, or partially human immunoglobulin loci. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, e.g., XenoMouse™, Green et al., Nature Genetics 7:13-21 (1994), US 2003-0070185, U.S. Pat. No. 5,789,650, and WO 96/34096.

Non-human antibodies to VLA-4 can also be produced, e.g., in a rodent. The non-human antibody can be humanized, e.g., as described in U.S. Pat. No. 6,602,503, EP 239 400, U.S. Pat. No. 5,693,761, and U.S. Pat. No. 6,407,213.

EP 239 400 (Winter et al.) describes altering antibodies by substitution (within a given variable region) of their complementarity determining regions (CDRs) for one species with those from another. CDR-substituted antibodies are predicted to be less likely to elicit an immune response in humans compared to true chimeric antibodies because the CDR-substituted antibodies contain considerably less non-human components. (Riechmann et al., 1988, Nature 332:323-327; Verhoeyen et al., 1988, Science 239:1534-1536). Typically, CDRs of a murine antibody substituted into the corresponding regions in a human antibody by using recombinant nucleic acid technology to produce sequences encoding the desired substituted antibody. Human constant region gene segments of the desired isotype (usually gamma I for CH and kappa for CL) can be added and the humanized heavy and light chain genes are co-expressed in mammalian cells to produce soluble humanized antibody.

Queen et al., Proc. Natl. Acad. Sci. USA 86:10029-10033 (1989) and WO 90/07861 have described a process that includes choosing human V framework regions by computer analysts for optimal protein sequence homology to the V region framework of the original murine antibody, and modeling the tertiary structure of the murine V region to visualize framework amino acid residues which are likely to interact with the murine CDRs. These murine amino acid residues are then superimposed on the homologous human framework. See also U.S. Pat. Nos. 5,693,762; 5,693,761; 5,585,089; and 5,530,101. Tempest et al. (1991, Biotechnology 9:266-271) utilize, as standard, the V region frameworks derived from NEWM and REI heavy and light chains respectively for CDR-grafting without radical introduction of mouse residues. An advantage of using the Tempest et al., approach to construct NEWM and REI based humanized antibodies is that the 3-dimensional structures of NEWM and REI variable regions are known from x-ray crystallographic studies, and thus specific interactions between CDRs and V region framework residues can be modeled.

Non-human antibodies can be modified to include substitutions that insert human immunoglobulin sequences, e.g., consensus human amino acid residues at particular positions, e.g., at one or more of the following positions (preferably at least five, ten, twelve, or all): (in the FR of the variable domain of the light chain) 4L, 35L, 36L, 38L, 43L, 44L, 58L, 46L, 62L, 63L, 64L, 65L, 66L, 67L, 68L, 69L, 70L, 71L, 73L, 85L, 87L, 98L, and/or (in the FR of the variable domain of the heavy chain) 2H, 4H, 24H, 36H, 37H, 39H, 43H, 45H, 49H, 58H, 60H, 67H, 68H, 69H, 70H, 73H, 74H, 75H, 78H, 91H, 92H, 93H, and/or 103H (according to the Kabat numbering). See, e.g., U.S. Pat. No. 6,407,213.

Fully human monoclonal antibodies that bind to VLA-4 can be produced, e.g., using in vitro-primed human splenocytes, as described by Boerner et al., 1991, J. Immunol., 147, 86-95. They may be prepared by repertoire cloning as described by Persson et al., 1991, Proc. Nat. Acad. Sci. USA 88:2432-2436; or by Huang and Stollar, 1991, J. Immunol. Methods 141:227-236; U.S. Pat. No. 5,798,230. Large nonimmunized human phage display libraries may also be used to isolate high affinity antibodies that can be developed as human therapeutics using standard phage technology (see, e.g., Vaughan et al. (1996) Nat. Biotech. 3:309-314; Hoogenboom et al. (1998) Immunotechnology 4:1-20; and Hoogenboom et al. (2000) Immunol Today 2:371-8; US 2003-0232333).

### Antibody Production

Antibodies can be produced in prokaryotic and eukaryotic cells. In one embodiment, the antibodies (e.g., scFv's) are expressed in a yeast cell such as Pichia (see, e.g., Powers et al. (2001) J. Immunol. Methods 251:123-35), Hanseula, or Saccharomyces.

In one embodiment, antibodies, particularly full length antibodies, e.g., IgG's, are produced in mammalian cells. Exemplary mammalian host cells for recombinant expression include Chinese Hamster Ovary (CHO cells) (including dhfr- CHO cells, described in Urlaub et all (1980) Proc. Natl. Acad. Sci. USA 77:4216-4220, used with a DHFR selectable marker, e.g., as described in Kaufman et al. (1982) Mol. Biol. 159:601-621), lymphocytic cell lines, e.g., NS0 myeloma cells and SP2 cells, COS cells, K562, and a cell from a transgenic animal, e.g., a transgenic mammal. For example, the cell is a mammary epithelial cell.

In addition to the nucleic acid sequence encoding the immunoglobulin domain, the recombinant expression vectors may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017). Exemplary selectable marker genes include the dihydrofolate reductase (DHFR) gene (for use in dhfr⁻ host cells with methotrexate selection/amplification) and the neo gene (for G418 selection).

In an exemplary system for recombinant expression of an antibody (e.g., a full length antibody or an antigen-binding portion thereof), a recombinant expression vector encoding both the antibody heavy chain and the antibody light chain is introduced into dhfr- CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. For example, some antibodies can be isolated by affinity chromatography with a Protein A or Protein G. U.S. Pat. No. 6,602,503 also describes exemplary methods for expressing and purifying a VLA-4 binding antibody.

Antibodies may also include modifications, e.g., modifications that alter Fc function, e.g., to decrease or remove interaction with an Fc receptor or with Clq, or both. For example, the human IgG1 constant region can be mutated at one or more residues, e.g., one or more of residues 234 and 237, e.g., according to the numbering in U.S. Pat. No. 5,648,260. Other exemplary modifications include those described in U.S. Pat. No. 5,648,260.

For some antibodies that include an Fc domain, the antibody production system may be designed to synthesize antibodies in which the Fc region is glycosylated. For example, the Fc domain of IgG molecules is glycosylated at asparagine 297 in the CH2domain. This asparagine is the site for modification with biantennary-type oligosaccharides. This glycosylation participates in effector functions mediated by Feγ receptors and complement Clq (Burton and Woof (1992) Adv. Immunol. 51:1-84; Jefferis et al. (1998) Immunol. Rev. 163:59-76). The Fc domain can be produced in a mammalian expression system that appropriately glycosylates the residue corresponding to asparagine 297. The Fc domain can also include other eukaryotic post-translational modifications.

Antibodies can also be produced by a transgenic animal. For example, U.S. Pat. No. 5,849,992 describes a method for expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly.

Antibodies can be modified, e.g., with moiety that improves its stabilization and/or retention in circulation, e.g., in blood, serum, lymph, bronchoalveolar lavage, or other tissues, e.g., by at least 1.5, 2, 5, 10, or 50 fold.

For example, a VLA-4 binding antibody can be associated with a polymer, e.g., a substantially non-antigenic polymers, such as polyalkylene oxides or polyethylene oxides. Suitable polymers will vary substantially by weight. Polymers having molecular number average weights ranging from about 200 to about 35,000 (or about 1,000 to about 15,000, and 2,000 to about 12,500) can be used.

For example, a VLA-4 binding antibody can be conjugated to a water soluble polymer, e.g., hydrophilic polyvinyl polymers, e.g. polyvinylalcohol and polyvinylpyrrolidone. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene (Pluronics); polymethacrylates; carbomers; branched or unbranched polysaccharides which comprise the saccharide monomers D-mannose, D- and L-galactose, fucose, fructose, D-xylose, L-arabinose, D-glucuronic acid, sialic acid, D-galacturonic acid, D-mannuronic acid (e.g. polymannuronic acid, or alginic acid), D-glucosamine, D-galactosamine, D-glucose and neuraminic acid including homopolysaccharides and heteropolysaccharides such as lactose, amylopectin, starch, hydroxyethyl starch, amylose, dextrane sulfate, dextran, dextrins, glycogen, or the polysaccharide subunit of acid mucopolysaccharides, e.g. hyaluronic acid; polymers of sugar alcohols such as polysorbitol and polymannitol; heparin or heparon.

### Pharmaceutical Compositions

A VLA-4 blocking agent, such as a VLA-4 binding antibody (e.g., natalizumab), can be formulated as a pharmaceutical composition. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible.

A “pharmaceutically acceptable salt” refers to a salt that retains the desired biological activity of the parent compound and does not impart any undesired toxicological effects (see, e.g., Berge et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

Natalizumab and other agents described herein can be formulated according to standard methods. Pharmaceutical formulation is a well-established art, and is further described in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7^{th} Ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X).

In one embodiment, a VLA-4 blocking agent, e.g., VLA-4 binding agent, e.g., natalizumab can be formulated with excipient materials, such as sodium chloride, sodium dibasic phosphate heptahydrate, sodium monobasic phosphate, and polysorbate 80. It can be provided, for example, in a buffered solution at a concentration of about 20 mg/ml and can be stored at 2-8° C. Natalizumab (TYSABRI®) can be formulated as described on the manufacturer's label.

Pharmaceutical compositions may also be in a variety of other forms. These include, for example, liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application. Typically compositions for the agents described herein are in the form of injectable or infusible solutions.

Such compositions can be administered by a parenteral mode (e.g., intravenous, subcutaneous, intraperitoneal, intramuscular injection). The phrases “parenteral administration” and “administered parenterally” as used herein mean modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Pharmaceutical compositions typically must be sterile and stable under the conditions of manufacture and storage. A pharmaceutical composition can also be tested to insure it meets regulatory and industry standards for administration.

The composition can be formulated as a solution, microemulsion, dispersion, liposome, or other ordered structure suitable to high drug concentration. Sterile injectable solutions can be prepared by incorporating the active compound in the required amount into an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

### Administration

A VLA-4 blocking agent, e.g., VLA-4 binding antibody, e.g., natalizumab, can be administered to a subject, e.g., a human subject, by a variety of methods. For many applications, the route of administration is one of: intravenous injection or infusion, subcutaneous injection, or intramuscular injection. A VLA-4 blocking agent, e.g., VLA-4 binding antibody, such as natalizumab, can be administered as a fixed dose, or in a mg/kg dose, but preferably as a fixed dose. The antibody can be administered intravenously (IV) or subcutaneously (SC).

The VLA-4 blocking agent, e.g., VLA-4 binding antibody, e.g., natalizumab, can be administered at a fixed unit dose of between 50-1000 mg IV, e.g., between 100-600 mg IV, e.g., about 300 mg IV. A unit dose can be administered every 4 weeks or less or more frequently, e.g., every 2 weeks or weekly. When administered subcutaneously, the antibody is typically administered at a dose between 50-100 mg SC (e.g., 75 mg), e.g., at least once a week (e.g., twice a week). It can also be administered in a bolus at a dose of between 1 and 10 mg/kg, e.g., about 6.0, 4.0, 3.0, 2.0, 1.0 mg/kg. In some cases, continuous administration may be indicated, e.g., via a subcutaneous pump.

The dose can also be chosen to reduce or avoid production of antibodies against the VLA-4 binding antibody, to achieve greater than 40, 50, 70, 75, or 80% saturation of the α4 subunit, to achieve to less than 80, 70, 60, 50, or 40% saturation of the α4 subunit, or to prevent an increase the level of circulating white blood cells.

Moreover, subjects who do not have clinically definite multiple sclerosis may be administered a reduced dose of a VLA-4 blocking agent, e.g., VLA-4 binding antibody, e.g., natalizumab, relative to subjects who have clinically definite multiple sclerosis. For example, subjects who are at risk, but do not have clinically definite multiple sclerosis can receive a VLA-4 blocking agent, e.g., VLA-4 binding antibody, e.g., natalizumab, at a fixed unit dose of between 20-300 mg IV, e.g., 20-150 mg IV (e.g., every four weeks), or between 20-70 or 20-40 mg SC (e.g., about 35 mg), e.g., at least one a week.

In certain embodiments, the active agent may be prepared with a carrier that will protect the compound against rapid release, such as a controlled release formulation, including implants, pumps, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York, 1978.

Pharmaceutical compositions can be administered with medical devices. For example, pharmaceutical compositions can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Of course, many other such implants, delivery systems, and modules are also known.

Dosage regimens can be adjusted to provide a desired response, e.g., a therapeutic response or a combinatorial therapeutic effect. Dosage unit form or “fixed dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with the other agent.

A pharmaceutical composition may include a “therapeutically effective amount” of an agent described herein. A therapeutically effective amount of an agent may vary according to factors such as the disease state, age, sex, and weight of the individual, and the ability of the compound to elicit a desired response in the individual, e.g., modulation of a risk factor, delay of onset or attenuation of severity a clinical episode of neurologic deficit, amelioration of at least one disorder parameter, e.g., a multiple sclerosis parameter, or amelioration of at least one symptom of the disorder, e.g., multiple sclerosis. A therapeutically effective amount is also one in which any toxic or detrimental effects of the composition is outweighed by the therapeutically beneficial effects.

Combination Therapy

In certain embodiments, a subject, e.g., a subject who has risk for multiple sclerosis, e.g., as described herein, can be administered a second agent, in combination with a VLA-4 blocking agent, e.g., VLA-4 binding antibody, e.g., natalizumab. Non-limiting examples of agents for treating or preventing multiple sclerosis which can be administered with a VLA-4 blocking agent include agents described in co-pending application, U.S. Ser. No. 60/603,468, filed Aug. 20, 2004, attorney docket number 10287-087P01/P0608, titled “Combination Therapy.”

All patent applications, patents, references and publications included herein are incorporated herein by reference.

Other embodiments are within the scope of the following claims.

1. A method of treating a subject at risk for relapsing or progressive multiple sclerosis, the method comprising administering to the subject a VLA-4 binding antibody.

2. The method of claim 1, wherein the subject has experienced one clinical episode of focal neurologic deficit.

3. The method of claim 2, wherein the antibody is administered within 4 weeks of the clinical episode.

4. The method of claim 2, wherein the neurologic deficit is evidenced by one or more symptoms selected from the group consisting of: weakness of one or more extremities, paralysis of one or more extremities, tremor of one or more extremities, uncontrollable muscle spasticity, sensory loss or abnormality, decreased coordination, loss of balance, loss of ability to think, abstractly, loss of ability to generalize, difficulty speaking, difficulty understanding speech, monocular or binocular visual loss, and bladder or bowel discontrol.

5. The method of claim 1, wherein the subject has had a cranial scan showing physical evidence of brain tissue inflammation or myelin sheath damage.

6. The method of claim 5, wherein the cranial scan is selected from the group consisting of: a radiographic scan, a computed tomography (CT) scan, and a magnetic resonance imaging (MRI) scan.

7. The method of claim 5, wherein the subject has between 1 and 50 individual brain lesions detectable by MRI.

8. The method of claim 1, wherein the subject has serum antibodies against one or both of myelin oligodendrocyte glycoprotein (MOG) and myelin basic protein (MBP).

9. The method of claim 5, wherein the subject has experienced one clinical episode of neurologic deficit.

10. The method of claim 5, wherein the subject has not experienced a clinical episode of neurologic deficit.

11. The method of claim 8, wherein the subject has experienced one clinical episode of neurologic deficit.

12. The method of claim 8, wherein the subject has not experienced a clinical episode of neurologic deficit.

13. The method of claim 1, wherein the subject has not experienced a clinical episode of focal neurologic deficit and has one or more of the following characteristics:
(a) has a plurality of brain lesions or scars greater than or equal to 3 mm in size detectable by cranial scan,
(b) has serum antibodies against one or both of myelin oligodendrocyte glycoprotein (MOG) and myelin basic protein (MBP),
(c) has increased, levels of CSF IgG compared to a negative control, and
(d) has elevated levels of myelin basic protein (MBP) compared to a negative control.

14. The method of claim 1, wherein the subject has experienced one clinical episode of focal neurologic deficit and has one or more of the following characteristics:
(a) has a plurality, of brain lesions or sears greater than or equal to 3 mm in size detectable by cranial scan,
(b) has serum antibodies against one or both of myelin oligodendrocyte glycoprotein (MOG) and myelin basic protein (MBP),
(c) has increased levels of CSF IgG compared to a negative control, and
(d) has elevated levels of myelin basic protein (MBP) compared to a negative control.

15. The method of claim 1, further comprising, before the administering step, selecting a subject as being at risk for MS on the basis of one or more of: (a) cranial scan evidence of myelin sheath damage, (b) presence of serum antibodies against one or both of MOG and MBP, (c) presence of increased levels of CSF IgG, (d) presence of elevated levels of MBP, and (c) occurrence of one clinical episode of focal neurologic deficit.

16. The method of claim 1, wherein the subject has a family history of multiple sclerosis.

17. The method of claim 1, wherein the subject has had one acute isolated demyelinating event involving the optic nerve, spinal cord or cerebellum.

18. The method of claim 1, wherein the subject has a plurality of clinically silent brain MRI lesions greater than or equal to 3 mm in size.

19. The method of claim 1, wherein the subject has transverse myelitis.

20. The method of claim 1, wherein the subject has optic neuritis.

21. A method of treating a subject, the method comprising:
performing a scan on a subject, and
administering to the subject a VLA-4 binding antibody if the scan shows evidence of clinically silent brain tissue inflammation or myelin sheath damage.

22. A method of treating a subject for a monophasic demyelinating disorder, the method comprising: identifying a subject having a monophasic demyelinating disorder; and administering to the subject a VLA-4 binding antibody.

23. The method of claim 22, wherein the subject has transverse myelitis.

24. The method of claim 22, wherein the subject has optic neuritis.

25. The method of claim 22, wherein the subject has acute disseminated encephalomyelitis (ADEM).

26. The method of claim 1, wherein the VLA-4 binding antibody binds at least the α chain of VLA-4.

27. The method of claim 1, wherein the VLA-4 binding antibody comprises natalizumab.

28. The method of claim 1, wherein the VLA-4 binding antibody competes with HP1/2 or natalizumab for binding to VLA-4.

29. The method of claim 1, wherein the VLA-4 binding antibody is human or humanized.

30. The method of claim 1, wherein the VLA-4 binding antibody is administered in combination with a second therapeutic agent.
